(19) United States
(12) Patent Application Publication
(10) Pub. No.: US 2008/0194631 A1
(43) Pub. Date: Aug. 14, 2008

(54) Medicament For the Treatment of Central Nervous System Disorders

(75) Inventors: Fabrice Trovero, Avaray (FR); Philippe Bernard, Orleans (FR)

Correspondence Address: OLIFF & BERRIDGE, PLC, P.O. BOX 320850, ALEXANDRIA, VA, 22320-4850, US

(73) Assignees: Key OBS, Orleans (FR); Greenpharma, Saint Beauzire (FR)

(21) Appl. No.: 11/658,643

(22) PCT Filed: Jul. 26, 2005
(86) PCT No.: PCT/FR05/01942

§ 371 Date: Mar. 13, 2007

(30) Foreign Application Priority Data
Jul. 26, 2004 (FR) 0408257

(51) Int. Cl. A61K 31/451; A61P 25/36; A61P 25/18; A61P 25/24

(52) U.S. Cl. 514/325; 514/317

(57) ABSTRACT

The invention relates to the use of a molecule, alone or in combination, chosen from the group of molecules having a simultaneous antagonist action on the alpha1-noradrenergic, glutamatergic NMDA and serotoninergic 5HT2 receptors, for the preparation of a medicament intended for treating pathologies of the central nervous system.

The pharmacological treatment of dependence is a major public health challenge. For a long time oriented toward help with withdrawal in opiate addiction, this type of treatment is also necessary for preventing the abusive consumption of other products such as psychostimulants, tobacco or alcohol.

The current pharmacopoeia has therefore developed around substitute products (nicotine patches, opiate analogues and the like) whose principal mode of action is to limit the physical symptoms induced by withdrawal. Nevertheless, there are very few substances intended to prevent the states of psychological dependence, that is to say the irrepressible need or craving for, on the product. This state of dependence is much stronger and quite obviously represents the major factor for a relapse.

### Current Treatments

### Heroin Addiction

The treatment of the physical signs of withdrawal is the first management in the disintoxication cure. The substitute products may be used with doses adapted to prevent the symptoms of withdrawal.

### Clonidine

The product most widely used for this purpose is clonidine, an agonist of the alpha2-adrenergic receptors which is generally used in the treatment of hypertension. Its action on the alpha2-adrenergic receptors reduces the hyperexcitability of the noradrenergic neurons and consequently limits the effects of stopping the taking of opiates on the autonomous system.

### Methadone and Levomethadyl Acetate

Methadone and levomethadyl acetate are two agonists of the type μopiate receptors. Because of a slow kinetics of action, methadone causes high dependence. One of the advantages of this type of treatment is to obtain a “controlled” consumption of an opoid substance.

### Buprenorphine

It is a partial agonist of the μ type opiate receptors and an agonist of the □ type receptors. Stopping the treatment causes a withdrawal syndrome. The product exists as tablets (Temgesic®, treatment of pain) and grinding the tablets can give rise to intravenous administrations, which are sought by the addict in order to increase the euphoria-causing effects. The Temgesic® form has been replaced by a “high dosage” galenic form, Subutex®.

### Treatment of Nicotine Addiction

Nicotine substitution via patches or chewing gums is the only replacement therapy which has shown some efficacy.

Bupropion was recently proposed in the treatment of nicotine addiction. It is an antidepressant which is thought to act by increasing the release of noradrenaline without modifying the recapture thereof. Bupropion is also an inhibitor of dopamine recapture, which could explain its anti-craving properties. It should also be noted that bupropion is an antagonist of the central nicotine receptors, which could also contribute to its efficacy. However, clinical studies do not appear to indicate a high therapeutic effect.

Treatment of Drug Addiction with Psychostimulants

Cocaine causes a high psychological dependence against which no medicament has yet shown real efficacy. Some lines of approach have been proposed. The opiate agonists used in heroin withdrawal (buprenorphine and methadone) have been tested for help with cocaine withdrawal. The problems linked to the consumption of crack are treated mainly with anxiolytics and sedative neuroleptics in order to prevent anxiety and hyperexcitability states.

### Treatment of Alcoholism

Pharmacological treatments are proposed in the treatment of alcoholism. However, there is currently no satisfactory treatment.

### Opiate Antagonists

The opoid systems play a key role in the regulation of the consumption behavior by contributing to the reinforcing effects of alcohol intake. It should be recalled that the opiates stimulate the activity of the dopaminergic systems. Naltrexone, an opiate antagonist, has been tested in clinical trials. Studies have then shown that naltrexone reduced alcohol intake, the relapse rate and the desire to drink, in particular in the case of serious alcoholization. It is therefore the first pharmacological agent against alcoholism which acts otherwise than by triggering a phenomenon of aversion.

Unfortunately, the use of naltrexone is limited because of its side effects at the gastrointestinal level (nausea, vomiting, loss of appetite) (O'Malley et al., 1992; Volpicelli et al., 1992; Kranzler et al., 2000)

### Naltrindole

It is an antagonist of the 6 type opiate receptors, which shows some efficacy in animal models.

### Acamprosate

Even though its mechanism of action has not been completely elucidated, a bundle of arguments makes it possible to think that the action of acamprosate involves modulation of glutamatergic transmission. It would appear that the molecule is effective, at least in the treatment of withdrawal symptoms. Its efficacy against craving for alcohol is still debated.

### Serotoninergic Antidepressants

The serotoninergic transmission also plays an important role in the pathophysiology of alcohol dependence. Inhibitors of serotonin recapture are antidepressants which have been tested in the treatment of alcoholism (Naranjo et al., 1984, 1987 and 1990). Therapeutic trials using these serotoninergic substances have given variable results, clinical studies not demonstrating real efficacy.

### Benzodiazepines

Benzodiazepines are the medicaments most widely used for alcohol withdrawal (for a review, Lejoyeux et al., 1998). Benzodiazepines are used with efficacy at the time of withdrawal. This efficacy is discussed over the long term, all the more so since the patients follow this type of treatment in order to combat the symptoms of abstinence such as anxiety and insomnia. The question regarding the benefit/risk ratio exists since it involves replacing an abused product with another in a patient already sensitive to the phenomena of dependence.

### Aversive Medications

The first aversive medicament against alcohol was disulfiram, which has been used since 1940. When it is consumed simultaneously with alcohol, this product triggers unpleasant effects such as nausea, vomiting, an increase in blood pressure and in the heartbeat.

### State of Research

The current knowledge in neurobiology makes it possible to describe the major transmission systems involved in dependence states. In general, the key role of the ascendant dopaminergic systems is clearly accepted. These dopaminergic neurons have their cellular bodies in a deep cerebral structure, the ventral tegmental area (VTA). Their projections are directed toward the front of the brain, mainly in the frontal cortex and in the nucleus accumbens. Most of the substances which trigger pleasant, or even euphoria-producing, effects induce activation of these dopaminergic neurons. The repeated activation of these neurons is thought to trigger regulatory processes which are thought to be responsible for the state of dependence. The state of dependence is thought to be a reflection of a need for sustained activation of these neurons.

The search for substances which are active against dependence requires good knowledge of the underlying cerebral mechanisms of addiction. The use of experimental models in animals, in particular in rodents, makes this search possible. In animals, addictive substances trigger a locomotor hyperactivity. This hyperactivity is a very good experimental index of the motivational properties of the product. That is the case for psychostimulants such as cocaine and amphetamine. Other substances, which induce at certain doses sedative, hypnotic or relaxing effects, have more complicated behavior response profiles. That is the case for alcohol and opiates which exhibit biphasic response curves.

On the other hand, for all these products, the repetition of administrations at identical doses causes a gradual increase in the locomotor behavior response. This phenomenon is called “behavioral sensitization”. If it is considered that the reinforcing properties are the common denominator for these products, it is possible to accept that this phenomenon indeed reflects the motivational properties of a product. By contrast, other substances trigger sharply a locomotor hyperactivity, without a behavioral sensitization appearing after repeated administrations. These products are not reinforcing and do not cause abuse behavior.

All the products which trigger behavioral sensitization in animals exhibit reinforcing properties and are therefore potentially substances of abuse. It is interesting to observe that this experimental paradigm also makes it possible to reproduce other aspects of drug abuse in humans. Indeed, behavioral sensitization is a phenomenon which is maintained after withdrawal for very long periods and the experimental context triggering it reproduces factors which facilitate relapse (environment associated with the administration of the drug). The analogy with the drug abuse behavior in humans is completed by the fact that, in animals, self-administration of heroin also causes sensitization of the locomotor responses to heroin (De Vries et al., 1998).

It should be noted that behavioral sensitization represents the opposite of tolerance. Indeed, in this case, it is the increase in the doses of a product which makes it possible to obtain an equivalent physiological or behavioral response. The best known example of tolerance is that of the treatment of pain with morphine, where indeed, the progression of the pathology requires increasing the doses in order to control the pain (Colpaërt, 1997). In this case of treatment of pain, psychological dependence will not develop.

Numerous studies have been carried out using behavioral sensitization as an experimental model. However, the mechanisms which may explain the maintenance of the phenomenon in the long term (several months) are still not well known.

In general, behavioral sensitization was associated with the modification of the reactivity of the dopaminergic neurons located in the VTA and innervating the nucleus accumbens. Two elements were dissociated, the induction of sensitization, which appears in connection with events taking place in the VTA, and the expression of this sensitization, which appears to be linked more to the release of dopamine in the nucleus accumbens. Thus:
The repeat of injections of a psychoactive substance causes an increase in the extracellular levels of dopamine in the VTA. This effect is mediated by desensitization of the D2 type dopaminergic receptors. These phenomena could intervene at the time of the induction of sensitization.
The blocking of the D1 type dopaminergic receptors present on the glutamatergic and GABAergic ends prevents the induction of sensitization (Stewart & Vezina, 1989; Kalivas & Duffy, 1995; Vezina, 1996).
The blocking of the NMDA type glutamatergic receptors of the VTA prevents the induction of sensitization (Kalivas, 1995), the NMDA receptors of the nucleus accumbens are not brought into play (Vezina et al., 2000).
The prefrontal cortex and its projections at the level of the ATV play an important role (for a review see Wolf et al., 1998). Behavioral sensitization is thought to correspond to an increase in the sensitivity to the glutamate released in the VTA by the neurons derived from the prefrontal cortex (Tong et al., 1995). This increase is maintained for 20 days after stopping cocaine and does not appear in the presence of antagonists of the D1 type dopaminergic receptors (Kalivas & Duffy, 1998; Wolf & Xue, 1998).
As regards the behavioral sensitization induced by repeated administrations of opiates, it appears to be independent of the D1 and D2 receptors (Vezina & Stewart, 1989; Jeziorski & White, 1995), but, as is the case for psychostimulants, it calls into play the NMDA type glutamatergic receptors. The prefrontal cortex is also involved (Tzschentke & Schmidt, 2000).
The stimulation of the 5HT1A receptors, by reducing the inhibitory action of the serotoninergic transmission on the dopaminergic neurons, increases sensitization to cocaine. (De la Garza & Cunningham, 2000).
The selective blocking of the alpha1-adrenergic receptors reduces the effects of amphetamine (Blanc et al., 1994). These results indicate that the major neurotransmission systems interact during the development and the maintenance of behavioral sensitization. The activity of the glutamatergic, noradrenergic and serotoninergic systems appears to be particularly involved in the modulation of the dopaminergic systems underlying this phenomenon.

On the other hand, these effects were for the most part correlated with the observations made in other tests for addiction such as the place preference test and the self-administration of drugs test. Some medicaments of the antipsychotic (neuroleptic) class exhibit this feature of blocking various neurotransmission systems by acting in particular on the dopaminergic, noradrenergic and serotoninergic receptors. These products were tested in the drug addiction treatment mainly because of their capacity for thwarting the acute dopaminergic effects of psychostimulants such as cocaine. Unfortunately, the dopaminergic profile of these substances causes numerous side effects, in particular extrapyramidal effects.

These results show nevertheless the close relationship between an antipsychotic activity and an anti-dependence efficacy. This relationship may be attributed to the involvement of the same neurotransmission systems. All these data show that it is possible to modulate behavioral sensitization by acting pharmacologically on various neurotransmission systems.

It is possible that simultaneous blocking of several targeted neurotransmission systems results in a substantial reduction in the initiation and/or expression of behavioral sensitization. In other words, the combination of antagonists, in particular of the glutamatergic, noradrenergic and serotoninergic systems could exhibit a synergy of action at the central level.

This pharmacological action would have the consequence of significantly reducing the states of dependence. In the light of what was previously described on the efficacy of antipsychotic substances, it is not impossible that substances which are effective against dependence also exhibit potential antipsychotic properties. The absence of dopaminergic properties would then limit the extrapyramidal effects.

Thus, despite the research studies carried out and the treatments proposed, none makes it possible to satisfactorily treat the pathologies developed above without causing substantial side effects.

### THE INVENTION

The problems developed above were solved by the applicant by testing the effects of a combination of molecules targeting particularly the glutamatergic, serotoninergic and noradrenergic neurotransmission systems on behavioral sensitization to amphetamine.

The associated molecules are nonselective, pharmacologically well known and do not exhibit major side effects, they are chosen from commercially available medicaments.

The invention relates to the use of a molecule, alone or in combination, chosen from the group of molecules having a simultaneous antagonist action on the alpha1-noradrenergic, glutamatergic NMDA and serotoninergic 5HT2 receptors, for the preparation of a medicament intended for treating pathologies of the central nervous system.

It also relates to the use, as mentioned above, characterized in that the pathologies are chosen from pharmacodependences, psychosis, nicotine addiction, disorders linked to alcohol consumption, schizophrenia, acute and chronic psychotic states, dementia, mood disorders, attention disorders, sleep disorders, impulsivity disorders, hyperactivity, acute and chronic psychotic states, states of dependence on addictive substances, dependence on alcohol, dependence on psychostimulants, dependence on opiates, dependence on benzodiazepines, dependence on tobacco, dependence on gambling.

It also relates to the use, as mentioned above, characterized in that the group of molecules having a simultaneous antagonist action on the alpha1-noradrenergic, glutamatergic NMDA and serotoninergic 5HT2 receptors consists of the following molecules:

1-(1-naphthyl)piperazine, seganserin, olanzapine, sarprogelate, mirtazapine, aripiprazole, mepiprazole, 4-(4-fluorobenzoyl)-1-(4-phenylbutyl)piperidine, AMI-193, amperozide, cinanserin, clozapine, cyclobenzaprine, ketanserin, MDL 11,939, metergoline, methiothepin, methysergide, mianserin, N-desmethylclozapine, piperazine, pirenperone, ritanserin, RS 102221, sertindole, SB 200646, SB 204741, SDZ SER 082, spiperone, AH 11110, amosulalol, corynanthine, HEAT, naftopidil, niguldipine, bunazosin, dapiprazole, prazosin, tamsulosin, RS 100329, RS 17053, terazosin, WB 4101, urapidil, 5-methylurapidil, (−)-MK801, (+)-MK801, (+/−)-1-(1,2-diphenylethyl)piperidine, (2R,3S)-Chlorpheg, (R)-4-carboxyphenyglycine, (R)-CPP, (RS)-CPP, arcaine, CGP 37849, CGP 39551, CGS 19755, D-AP5, D-AP7, DL-AP5, DL-AP7, L-AP5, loperamide, LY 235959, memantine, SDZ 220-040, SDZ 220-581, synthalin, and their pharmacologically acceptable salts.

It also relates to the use, as mentioned above, characterized in that at least one of the molecules is Ifenprodil.

It also relates to the use, as mentioned above, characterized in that at least one of the molecules is cyproheptadine.

It also relates to the use, as mentioned above, characterized in that the molecules are administered at a daily dose of between 0.1 mg and 1000 mg per day.

It also relates to a pharmaceutical composition comprising at least two molecules chosen from the group of molecules having a simultaneous antagonist action on the alpha1-noradrenergic, glutamatergic NMDA and serotoninergic 5HT2 receptors consisting of the following molecules:

1-(1-naphthyl)piperazine, seganserin, olanzapine, sarprogelate, mirtazapine, aripiprazole, mepiprazole, 4-(4-fluorobenzoyl)-1-(4-phenylbutyl)piperidine, AMI-193, amperozide, cinanserin, clozapine, cyclobenzaprine, ketanserin, MDL 11,939, metergoline, methiothepin, methysergide, mianserin, N-desmethylclozapine, piperazine, pirenperone, ritanserin, RS 102221, sertindole, SB 200646, SB 204741, SDZ SER 082, spiperone, AH 11110, amosulalol, corynanthine, HEAT, naftopidil, niguldipine, bunazosin, dapiprazole, prazosin, tamsulosin, RS 100329, RS 17053, terazosin, WB 4101, urapidil, 5-methylurapidil, (−)-MK801, (+)-MK801, (+/−)-1-(1,2-diphenylethyl)piperidine, (2R, 3S)-Chlorpheg, (R)-4-carboxyphenyglycine, (R)-CPP, (RS)-CPP, arcaine, CGP 37849, CGP 39551, CGS 19755, D-AP5, D-AP7, DL-APS, DL-AP7, L-AP5, loperamide, LY 235959, memantine, SDZ 220-040, SDZ 220-581, synthalin, and their pharmacologically acceptable salts, combined with a pharmaceutically acceptable vehicle or excipient.

It also relates to a pharmaceutical composition, characterized in that the content of molecule in the composition is between 0.1 mg and 1000 mg.

It also relates to a pharmaceutical composition, characterized in that the combined molecules are Ifenprodil and cyproheptadine.

### Experimental Results:

Among the molecules tested, the results obtained with ifenprodil and cyproheptadine are presented below.

Review of the Molecules:

Cyproheptadine: Periactin®

Symptomatic treatment of various allergic manifestations, 4 to 20 mg/day.

Antihistamine and antiserotoninergic activity.

Cyproheptadine was selected for its antiserotoninergic activity, in particular for its affinity for the 5HT2 and 5HT1C receptors.

Ifenprodil: Vadilex®

Treatment of the painful manifestations of arteriopathies during an ischemic attack, neuroprotective agent. 5 to 15 mg/day.

Ifenprodil was selected for its antagonist properties toward the NMDA and alpha1-noradrenergic receptors.

These two molecules are therefore particularly well described and pharmacological data in animals are available. There may be mentioned those which are directly linked to our field of study:

Cyproheptadine: no effect on the locomotor activity at 2 mg/kg in mice (Semenova and Tiku, 1997; Costall et al., 1998). Cyproheptadine antagonizes the hyper-locomotor effect of opiates (Gurtu, 1990).

Ifenprodil has anxiolytic effects (Fraser et al., 1996). Ifenprodil reduces the stimulant effects of alcohol, but does not block the expression of sensitization (Broadbent 2003). Ifenprodil reduces the physical signs induced by alcohol withdrawal (Malinowska et al., 1999).

Separately, under some experimental conditions, these two molecules reduce the acute locomotor effects of addictive substances without, as a result, having an obvious effect on the development of sensitization after repeated administrations.

The results obtained with these two molecules separately on the locomotor hyperactivity induced by amphetamine and then in combination on the development of sensitization induced after repeated administrations are presented below.

This example, described more precisely below, is not limiting.

The other molecules which make it possible to obtain similar results are chosen from the group consisting of the following molecules:

1-(1-naphthyl)piperazine, seganserin, olanzapine, sarprogelate, mirtazapine, aripiprazole, mepiprazole, 4-(4-fluorobenzoyl)-1-(4-phenylbutyl)piperidine, AMI-193, amperozide, cinanserin, clozapine, cyclobenzaprine, ketanserin, MDL 11,939, metergoline, methiothepin, methysergide, mianserin, N-desmethylclozapine, piperazine, pirenperone, ritanserin, RS 102221, sertindole, SB 200646, SB 204741, SDZ SER 082, spiperone, AH 11110, amosulalol, corynanthine, HEAT, naftopidil, niguldipine, bunazosin, dapiprazole, prazosin, tamsulosin, RS 100329, RS 17053, terazosin, WB 4101, urapidil, 5-methylurapidil, (−)-MK801, (+)-MK801, (+/−)-1-(1,2-diphenylethyl)piperidine, (2R, 3S)-Chlorpheg, (R)-4-carboxyphenyglycine, (R)-CPP, (RS)-CPP, arcaine, CGP 37849, CGP 39551, CGS 19755, D-AP5, D-AP7, DL-APS, DL-AP7, L-AP5, loperamide, LY 235959, memantine, SDZ 220-040, SDZ 220-581, synthalin, and their salts.

### EXAMPLE 1

### Materials

The principle of the behavioral sensitization test is based on the measurement of the locomotor activity. The latter is measured in the open-field test.

### Motor and Exploratory Activity Test in Open-Field

The animals used are 9-week old C57/BL6J male mice at the beginning of the experiment.

The open-fields used to measure the activity are 4 square chambers made of transparent plexiglas. Each open-field is arranged in a setting equipped with photoelectric cells which make it possible to record the movements of the animal.

### Effects of the Products on the Locomotor Activity

The products used have known psychotropic properties. At some doses, they can induce hypolocomotor effects. Since the principle of the study is based on the measurement of the locomotor activity, it is preferable to have a preliminary evaluation of these effects under the experimental conditions used. The first step of the protocol consists in measuring the effects of the products on the locomotor activity, in order to establish a range of doses which do not cause a major incapacitating effect.

### Products:

All the products are dissolved in physiological saline (0.9% NaCl) so that the volume for injection is 10 ml/kg of body weight.

IFENPRODIL was tested at the following doses: 1 mg/kg; 3 mg/kg and 10 mg/kg.

CYPROHEPTADINE was tested at the following doses: 0.3 mg/kg; 1 mg/kg; 3 mg/kg and 10 mg/kg.

### Summary of the Results on the Locomotor Activity:

Ifenprodil alone does not have a significant effect on locomotion at doses of less than 10 mg/kg.

Cyproheptadine alone starts to have a significant effect at doses greater than 3 mg/kg.

These results indicate that it is preferable not to use higher doses in the remainder of the experiments.

The experiments were also performed in order to evaluate the effect of the combination of the products:

IFENPRODIL (3 mg/kg)+CYPROHEPTADINE (1 mg/kg): reduction
IFENPRODIL (3 mg/kg)+CYPROHEPTADINE (0.3 mg/kg): reduction
IFENPRODIL (1 mg/kg)+CYPROHEPTADINE (0.1 mg/kg): no effect
IFENPRODIL (0.3 mg/kg)+CYPROHEPTADINE (0.3 mg/kg): no effect

In the amphetamine sensitization protocol, the doses used for the combination of the products were chosen based on these first observations and then optimized during the protocol.

IFENPRODIL (0.3 mg/kg)+CYPROHEPTADINE (0.3 mg/kg)
IFENPRODIL (1 mg/kg)+CYPROHEPTADINE (1 mg/kg)
IFENPRODIL (3 mg/kg)+CYPROHEPTADINE (1 mg/kg)

### Behavioral Sensitization Protocol

During this protocol, the animals are treated with amphetamine. Groups receive beforehand an administration of one of the products or the combination of both.

### Protocol

Each passage is performed in the following manner:
t=0: placing in the open-field
t=15 min: 1st injection (NaCl, IFENPRODIL, CYPROHEPTADINE or IFENPRODIL+CYPROHEPTADINE)
t=30 min: 2nd injection (NaCl or amphetamine)
t=90 min: exit from the open-field

During the 1st session, the animals receive NaCl at the 1st and 2nd injection. The experiment is performed over several sessions, 2 to 10 days apart.

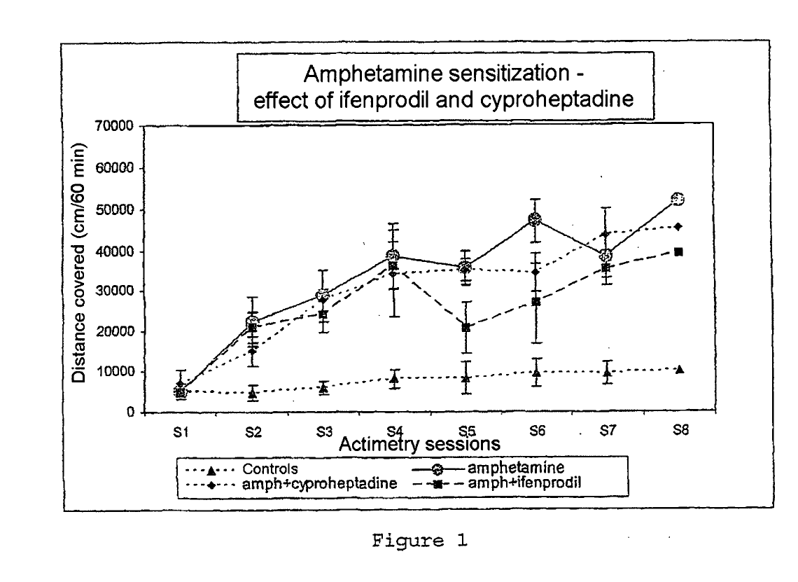

The experiments were performed on 5 groups of animals which received the following treatments at each session. The doses are indicated in FIGS. 1, 2 and 3.

| Group | 1st inj at T = 15′ | 2nd inj at T = 30′ |
|---|---|---|
| “Controls” | NaCl | NaCl |
| “Amphetamine” | NaCl | amphetamine |
| | | (2 mg/kg) |
| “Amphetamine + | Cyproheptadine | amphetamine |
| cyproheptadine” | | (2 mg/kg) |
| “Amphetamine + | Ifenprodil | amphetamine |
| ifenprodil” | | (2 mg/kg) |
| “Amphetamine + | Cyproheptadine + | amphetamine |
| ifenprodil + | Ifenprodil | (2 mg/kg) |
| cyproheptadine” | | |

### Results

FIG. 1: Effect of a treatment with ifenprodil and with cyproheptadine on the sensitization induced by amphetamine (n=4 animals per group)

Sessions 2 and 3: Ifenprodil 1 mg/kg and cyproheptadine 1 mg/kg

Next sessions: Ifenprodil 3 mg/kg and cyproheptadine 1 mg/kg

On the y-axis: Distance in cm (mean ±SEM)

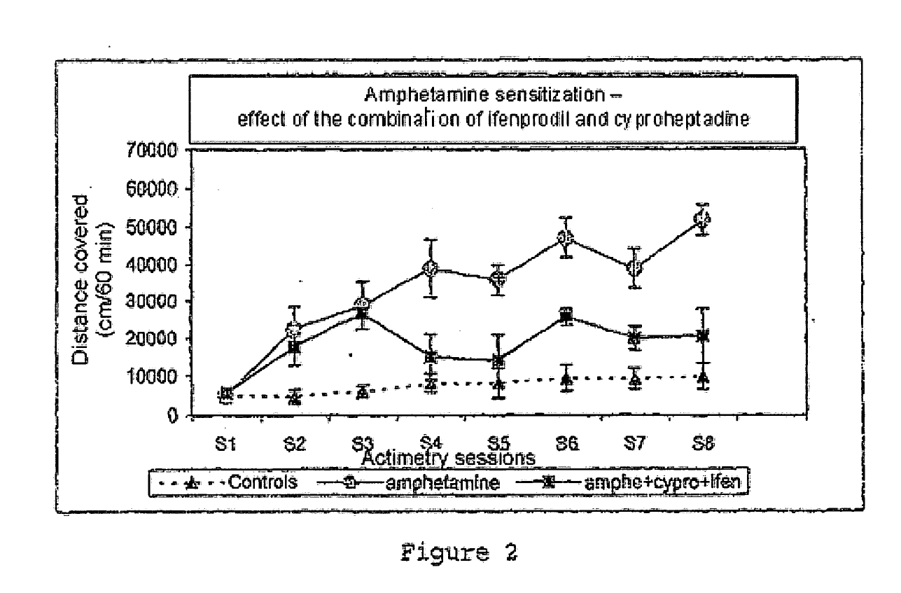

FIG. 2: Effect of a treatment with the combination ifenprodil+cyproheptadine on the sensitization induced by amphetamine (n=4 animals per group)

Sessions 2 and 3: Ifenprodil 1 mg/kg and cyproheptadine 1 mg/kg

Next sessions: Ifenprodil 3 mg/kg and cyproheptadine 1 mg/kg

On the y-axis: Distance in cm (mean ±SEM)

### Summary Table

Effect on the locomotor activity (cm covered, mean ±SEM) of the treatments alone and in combination at the first session and at the final sensitization session.

| Group | Session 2 | Session 11 |
|---|---|---|
| Controls | 4709 ± 1845 | 9934 ± 3405 |
| Amphetamine | 22 383 ± 6049 | 51 686 ± 3869 |
| Amphetamine + | 20 983 ± 3818 | 29 357 ± 4541 |
| Ifenprodil | | |
| Amphetamine + | 15 165 ± 3711 | 45 117 ± 10 900 |
| Cyproheptadine | | |
| Amphetamine + | 17 761 ± 4843 | 20 580 ± 7245 (*) |
| Ifenprodil + | | |
| Cyproheptadine | | |

(*) P < 0.05, significantly different from the amphetamine group.

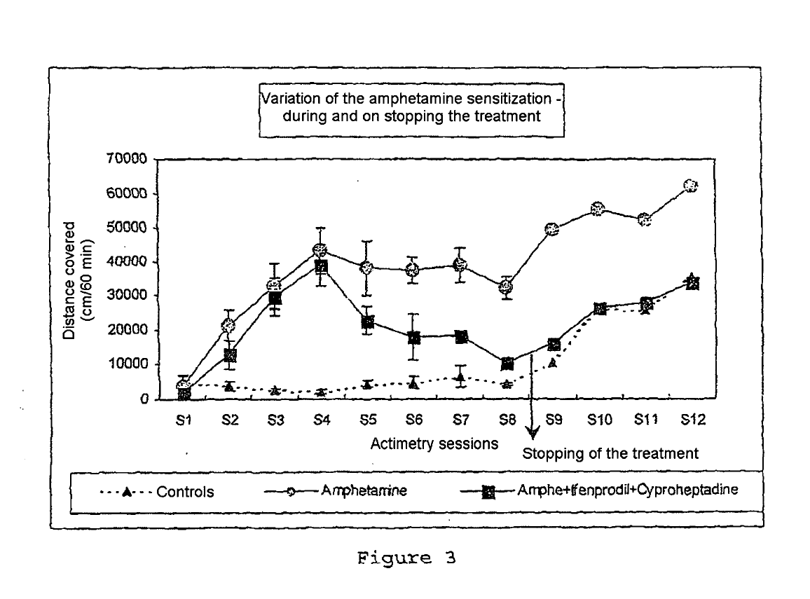

FIG. 3: Effect of a treatment with the combination ifenprodil+cyproheptadine during and on stopping the administrations of amphetamine (n=4 animals per group)

Sessions 2 to 4: Ifenprodil 0.3 mg/kg+cyproheptadine 0.3 mg/kg

Sessions 5 to 8: Ifenprodil 1 mg/kg+cyproheptadine 1 mg/kg

Sessions 9 to 12: Ifenprodil 0 mg/kg+cyproheptadine 0 mg/kg. The control group receives amphetamine like the other two groups.

On the y-axis: Distance in cm (mean ±SEM)

### Conclusion

At the doses used, the products injected separately or in combination have no significant effect on the hyperactivity induced by amphetamine.

Ifenprodil and cyproheptadine administered separately have limited effects on the development and the expression of the phenomenon of behavioral sensitization to amphetamine. The effect of ifenprodil is undoubtedly linked to its antagonist properties toward both the alpha1-adrenergic and NMDA type glutamatergic receptors. The effect of cyproheptadine is undoubtedly linked to its antagonist properties toward the serotoninergic receptors, in particular of the 5HT2 type. These effects are not long-lasting, they disappear after a repeated treatment.

Ifenprodil and cyproheptadine administered in combination significantly reduce the development of sensitization (p<0.05). It is the nonselective and simultaneous blocking of the alpha1-noradrenergic, NMDA-glutamatergic and 5HT2-serotoninergic receptors which is undoubtedly responsible for this action.

FIG. 3 shows that on stopping the treatment, the response to amphetamine is identical to that of the controls. The combination of the products therefore reduced the sensitization; it did not merely antagonize the locomotor effect of amphetamine.

These experiments show that ifenprodil and cyproheptadine administered in combination at doses which do not have a major incapacitating effect can very significantly reduce the sensitization induced by the psychostimulants. These results indicate that this combination of products can prevent the reinforcing effects of the various addictive substances, and consequently reduce the states of dependence.

Studies with other addictive substances (opiates, alcohol and the like) show useful results. They are used to evaluate the efficacy of the use according to the invention of molecules alone or in combination on other experimental models of addiction such as free consumption and place preference.

On the other hand, the efficacy of the use according to the invention of molecules alone or in combination on noradrenergic, glutamatergic and serotoninergic neurotransmission systems, and its effects on psychostimulants make this combination of this type of molecules novel and valuable antipsychotic substances.

### EXAMPLE 2

By the use according to the invention of molecules alone or in combination, a true effect in vivo was demonstrated for relatively variable contents which may be between 0.1 mg and 1000 mg in the pharmaceutical compositions. These values are in agreement with the effective doses known for each of the medicaments taken separately. The modes of administration and the galenic also correspond to the conventional modes of administration known to persons skilled in the art.

This use according to the invention of molecules alone or in combination allows the formulation of novel medicaments intended to treat or prevent pathologies of the central nervous system, in particular drug dependence, psychosis, nicotine addiction, disorders linked to alcohol consumption, schizophrenia, acute and chronic psychotic states, dementia, mood disorders, attention disorders, sleep disorders, impulsivity disorders, hyperactivity, acute and chronic psychotic states, states of dependence on addictive substances, dependence on alcohol, dependence on psychostimulants, dependence on opiates, dependence on benzodiazepines, disorders linked to gambling, dependence on tobacco.

### EXAMPLE 3

Given the pharmacological profile of the molecules chosen toward the noradrenergic and serotoninergic receptors, an antipsychotic activity was sought. The substances were tested in the model of locomotor hyperactivity induced by MK801. This model is particularly sensitive to atypical antipsychotics which exhibit good affinity for the serotoninergic and noradrenergic receptors. In this model and under our experimental conditions, clozapine (1 mg/kg) reduces by 56% the hyperactivity induced by MK 801. Clozapine is a reference atypical neuroleptic which has a very good affinity for the serotoninergic 5HT2 and noradrenergic alpha1 receptors.

The chosen molecules according to the invention demonstrated a true effect at the active doses in the preceding experiments (1 mg/kg ifenprodil+1 mg/kg cyproheptadine).

Effect of the combination 1 mg/kg ifenprodil+1 mg/kg cyproheptadine on the locomotor activity induced by MK801 (cm covered, mean +SEM).

| | Locomotor |
|---|---|
| Group | activity |
| Controls | 8854 ± 463 |
| MK 801 | 21 196 ± 1035 |
| MK 801 + Ifenprodil + | 15 740 ± 2668 (*) |
| cyproheptadine | |

(*) P < 0.05, significantly different from the MK 801 group.
(n = 6 animals per group)

The combination ifenprodil+cyproheptadine at the effective doses in the addiction model antagonizes at 45% the hyperactivity induced by MK801.

1. The use of a molecule, alone or in combination, chosen from the group of molecules having a simultaneous antagonist action on the alpha1-noradrenergic, glutamatergic NMDA and serotoninergic 5HT2 receptors, for the preparation of a medicament intended for treating pathologies of the central nervous system.

2. The use as claimed in claim 1, wherein the pathologies are chosen from pharmacodependences, psychosis, nicotine addiction, disorders linked to alcohol consumption, schizophrenia, acute and chronic psychotic states, dementia, mood disorders, attention disorders, sleep disorders, impulsivity disorders, hyperactivity, states of dependence on addictive substances, dependence on alcohol, dependence on psychostimulants, dependence on opiates, dependence on benzodiazepines, dependence on tobacco, dependence on gambling.

3. The use as claimed in claim 1, wherein the group of molecules having a simultaneous antagonist action on the alpha1-noradrenergic, glutamatergic NMDA and serotoninergic 5HT2 receptors consists of the following molecules: 1-(1-naphthyl)piperazine, seganserin, olanzapine, sarprogelate, mirtazapine, aripiprazole, mepiprazole, 4-(4-fluorobenzoyl)-1-(4-phenylbutyl)piperidine, AMI-193, amperozide, cinanserin, clozapine, cyclobenzaprine, ketanserin, MDL 11,939, metergoline, methiothepin, methysergide, mianserin, N-desmethylclozapine, piperazine, pirenperone, ritanserin, RS 102221, sertindole, SB 200646, SB 204741, SDZ SER 082, spiperone, AH 11110, amosulalol, corynanthine, HEAT, naftopidil, niguldipine, bunazosin, dapiprazole, prazosin, tamsulosin, RS 100329, RS 17053, terazosin, WB 4101, urapidil, 5-methylurapidil, (−)-MK801, (+)-MK801, (+/−)-1-(1,2-diphenylethyl)piperidine, (2R, 3S)-Chlorpheg, (R)-4-carboxyphenyglycine, (R)-CPP, (RS)-CPP, arcaine, CGP 37849, CGP 39551, CGS 19755, D-AP5, D-AP7, DL-APS, DL-AP7, L-AP5, loperamide, LY 235959, memantine, SDZ 220-040, SDZ 220-581, synthalin, and their pharmacologically acceptable salts.

4. The use as claimed in claim 1, wherein at least one of the molecules is Ifenprodil.

5. The use as claimed in claim 1, wherein at least one of the molecules is cyproheptadine.

6. The use as claimed in claim 1, wherein the molecules are administered at a daily dose of between 0.1 mg and 1000 mg per day.

7. A pharmaceutical composition comprising at least two molecules chosen from the group of molecules having a simultaneous antagonist action on the alpha1-noradrenergic, glutamatergic NMDA and serotoninergic 5HT2 receptors consisting of the following molecules:
1-(1-naphthyl)piperazine, seganserin, olanzapine, sarprogelate, mirtazapine, aripiprazole, mepiprazole, 4-(4-fluorobenzoyl)-1-(4-phenylbutyl)piperidine, AMI-193, amperozide, cinanserin, clozapine, cyclobenzaprine, ketanserin, MDL 11,939, metergoline, methiothepin, methysergide, mianserin, N-desmethylclozapine, piperazine, pirenperone, ritanserin, RS 102221, sertindole, SB 200646, SB 204741, SDZ SER 082, spiperone, AH 11110, amosulalol, corynanthine, HEAT, naftopidil, niguldipine, bunazosin, dapiprazole, prazosin, tamsulosin, RS 100329, RS 17053, terazosin, WB 4101, urapidil, 5-methylurapidil, (−)-MK801, (+)-MK801, (+/−)-1-(1,2-diphenylethyl)piperidine, (2R, 3S)-Chlorpheg, (R)-4-carboxyphenyglycine, (R)-CPP, (RS)-CPP, arcaine, CGP 37849, CGP 39551, CGS 19755, D-AP5, D-AP7, DL-APS, DL-AP7, L-AP5, loperamide, LY 235959, memantine, SDZ 220-040, SDZ 220-581, synthalin, and their pharmacologically acceptable salts, combined with a pharmaceutically acceptable vehicle or excipient.

8. The pharmaceutical composition as claimed in claim 7, wherein the content of molecule in the composition is between 0.1 mg and 1000 mg.

9. The pharmaceutical composition as claimed in claim 7, wherein the combined molecules are Ifenprodil and cyproheptadine.

10. The pharmaceutical composition as claimed in claim 9, wherein the content of molecule in the composition is between 0.1 mg and 1000 mg.
